(19) United States
(12) Patent Application Publication
(10) Pub. No.: US 2018/0072690 A1
(43) Pub. Date: Mar. 15, 2018

(54) Methods for Making Serotonin Reuptake Inhibitors

(71) Applicant: CIPLA Limited, Mumbai (IN)

(72) Inventors: Dharmaraj Ramachandra Rao, Maharashtra, Mumbai (IN); Geena Malhotra, Dadar (East), Mumbai (IN); Venkata Srinivas Pullela, Karnataka, Bangalore (IN); Shrikant Suresh Mudgal, Raigad, Maharashtra (IN)

(73) Assignee: CIPLA Limited, Mumbai (IN)

(21) Appl. No.: 15/561,621

(22) PCT Filed: Mar. 23, 2016
(86) PCT No.: PCT/GB2016/050822

§ 371 Date: Sep. 26, 2017

(30) Foreign Application Priority Data
Mar. 26, 2015 (IN) 1028/MUM/2015

(51) Int. Cl. C07D 295/096; C07D 211/20

(52) U.S. Cl. CPC C07D 295/096; C07D 211/20

(57) ABSTRACT

The present invention relates to a process for preparing serotonin reuptake inhibitors of formula (I) and pharmaceutically acceptable salts thereof, (Formula (I)) wherein, R₁, R₂, R₃, l, m, n and Z are as defined in the specification.

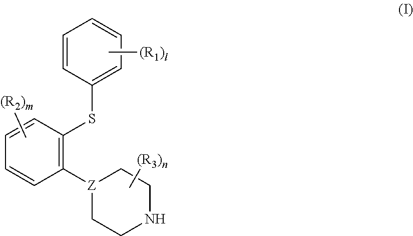

### TECHNICAL FIELD OF INVENTION

The present invention relates to an improved method for making serotonin reuptake inhibitors and pharmaceutically acceptable salts thereof.

### BACKGROUND OF INVENTION

Major depressive disorder (MDD) (also known as clinical depression, major depression, unipolar depression, or unipolar disorder; or as recurrent depression in the case of repeated episodes) is a mental disorder characterized by a pervasive and persistent low mood that is accompanied by low self-esteem and by a loss of interest or pleasure in normally enjoyable activities. Major depressive disorder is a disabling condition that adversely affects a person's family, work or school life, sleeping and eating habits, and general health. Episodes of depression often recur throughout a person's lifetime, although some may experience a single occurrence. In the United States, around 3.4% of people with major depression commit suicide, and up to 60% of people who commit suicide had depression or another mood disorder.

Selective serotonin reuptake inhibitors (SSRIs) are the primary medications prescribed in the treatment of major depressive disorder and anxiety disorders, owing to their relatively mild side-effects, and because they are less toxic in overdose than other antidepressants.

However, clinical studies on depression indicate that non-response to SSRIs is substantial, up to 30%. Another, often neglected, factor in antidepressant treatment is compliance, which has a rather profound effect on the patient's motivation to continue pharmacotherapy.

In order to circumvent some of these shortcomings of SSRI treatment, psychiatrists sometimes make use of augmentation strategies. Augmentation of antidepressants may be achieved e.g. by combination with mood stabilizers, such as lithium carbonate or triiodothyronin, or by the parallel use of electroshock.

It is known that a combination of inhibition of the serotonin transporter (SERT) with an activity on one or more serotonin receptors may be beneficial. The serotonin reuptake inhibitors are prescribed for the treatment of affective disorders such as depression, anxiety disorders including general anxiety disorder and panic disorder and obsessive compulsive disorder. Some of the compounds also have a combined effect of serotonin reuptake inhibition and 5-HT_{2C} receptor modulation.

WO 2003/029232 A1, incorporated by reference in its entirety, describes various serotonin reuptake inhibitors and pharmaceutically acceptable salts thereof per se, processes for their preparation as well as pharmaceutical compositions comprising the same. In particular, there are disclosed compounds of formula (I),

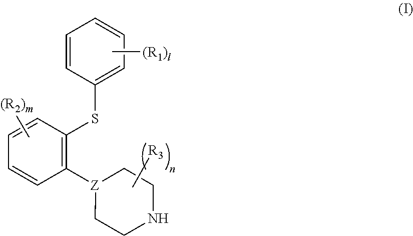

Wherein,
Z is N, C or CH;
Each R₁ and R₂ are independently selected from a group represented by hydrogen (—H), halogen, cyano, C₁₋₆-alkyl, C₁₋₆-alken, C₁₋₆-alkyn, C₁₋₆-alkenyl, C₁₋₆-alkynyl, C₁₋₆-alkyloxy, C₁₋₆-alkenyloxy, C₁₋₆-alkynyloxy, C₁₋₆-alkylylsulfanyl, C₁₋₆-alkenylsulfanyl, C₁₋₆-alkynylsulfanyl, hydroxy, hydroxy-C₁₋₆-alkyl, hydroxy-C₁₋₆-alken, hydroxy-C₁₋₆-alkyn, hydroxy-C₁₋₆-alkenyl, hydroxy-C₁₋₆-alkynyl, halo-C₁₋₆-alkyl, halo-C₁₋₆-alken, halo-C₁₋₆-alkyn, halo-C₁₋₆-alkenyl, halo-C₁₋₆-alkynyl, halo-C₁₋₆-alkyloxy, halo-C₁₋₆-alkenyloxy, halo-C₁₋₆-alkynyloxy, C₃₋₈-cycloalkyl, C₃₋₈-cycloalken, C₃₋₈-cycloalkenyl, C₃₋₈-cycloalk(en)yl-C₁₋₆-alk(en/yn) yl, C₁₋₆-alk (en/yn) ylsulfonyl, aryl, C₁₋₆-alkyloxycarbonyl C₁₋₆-alkenyloxycarbonyl C₁₋₆-alkynyloxycarbonyl, acyl, —NR₆CO—C₁₋₆-alkyl, —NR₆CO—C₁₋₆-alkenyl, —NR₆CO—C₁₋₆-alkynyl, CONR₆R_{7 or} or NR₆R₇;
Each R₃ is independently selected from the group represented by C₁₋₆-alkyl, or two R₃ attached to the same carbon atom may form a 3-6-membered spiro-attached cycloalkyl;

wherein each R₆ and R₇ is independently selected from the group represented by hydrogen, C₁₋₆-alkyl, C₁₋₆-alkenyl, C₁₋₆-alkynyl, C₃₋₈-cycloalkyl, C₃₋₈-cycloalkenyl, C₃₋₈-cycloalk(en)yl-C₁₋₆-alk(en/yn)yl, or aryl; or R₆ and R₇ together with the nitrogen to which they are attached form a 3-7-membered ring which optionally contains one further hetero atom; provided that both R₆ and R₇ are not hydrogen;

l is 0, 1, 2, 3, 4 or 5;

m is 0, 1, 2, 3 or 4;

n is 0, 1, 2, 3, 4, 5, 6, 7 or 8;

and acid addition salts thereof.

Among these compounds of formula (I) the following serotonin reuptake inhibitors are disclosed:

1-[2-(2,4-Dimethylphenylsulfanyl)phenyl]piperazine of formula IA:

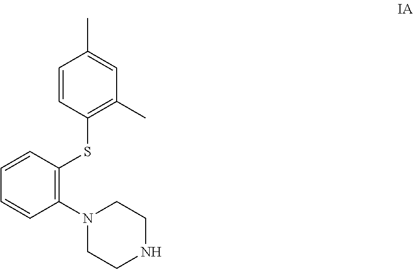 [SMILES: Cc1ccc(Sc2ccccc2N2CCNCC2)c(C)c1]

also known as vortioxetine, is a multimodal serotonergic compound intended to be used in the treatment of major depressive disorder (MDD) and generalized anxiety disorder. The compound shows antagonistic properties at 5-HT3A and 5-HT, receptors, partial agonistic properties at 5-HT1B receptors, agonistic properties at 5-HT receptors and potent serotonin reuptake inhibition via inhibition of the serotonin transporter (SERT).

4-{2-[(4-methylphenyl)sulfanyl]phenyl}piperidine of Formula IB:

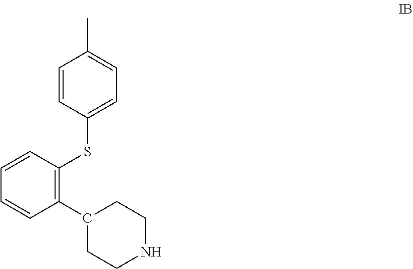 [SMILES: Cc1ccc(Sc2ccccc2C2CCNCC2)cc1]

also known as tedatioxetine (Lu AA24530) is being developed by Lundbeck for the treatment of MDD. It is a multimodal antidepressant and preclinical studies have shown that it acts as a monoamine enhancer with reuptake inhibition at monoamine transporters, as well as a 5-HT3 and 5-HT2C receptor antagonist.

The coupling of a thiophenol derivative with an aryl halide to obtain an aryl sulfide is one of the key steps in the synthesis of serotonin reuptake inhibitors of formula (I).

Several approaches are described in the literature to make carbon-sulfide bond formation.

The manufacturing process used to prepare vortioxetine and tedatioxetine was first disclosed in WO 03/029232. The process is based on solid-phase synthesis and exploits di-arene iron-assisted nucleophilic aromatic substitution reactions in a multistep process. In summary, 4-[piperazine-1-yl]carbonyloxymethyl]phenoxymethyl polystyrene was reacted with a di-arene iron salt, i.e. q-1,2-dichlorobenzene-q-cyclopentadienyliron(II) hexafluorophosphate followed by isolation and washing of the resin and further reaction with 2,4-dimethylthiophenol. Finally, the thus obtained resin was treated with 1,10-phenanthroline and light to de-complex cyclopentadienyliron. The overall yield was low, only 17%.

Several alternative palladium catalyzed processes for the preparation of aryl sulfides and vortioxetine are described in Examples 17 to 25 of WO 2007/144005 A1. These processes describe coupling of 2-bromoiodobenzene with 2,4-dimethylthiophenol in the presence of Pd₂(dba)₃ or Pd(dba)₂, t-BuOK and DPEphos in toluene or Pd(dba)₂, BINAP and t-BuONa in toluene yields 1-(2-bromophenylsulfanyl)-2,4-dimethylbenzene, which is alternatively prepared by coupling of either 1-iodo-2,4-dimethylbenzene or 1-bromo-2,4-dimethylbenzene with 2-bromobenzenethiol in the presence of Pd₂(dba)₃ and DPEphos.

In another approach, coupling of 2-bromoiodobenzene with N-Boc-piperazine in the presence of Pd2(dba)3 and xantphos yields aryl piperazine, which then condenses with 2,4-dimethylthiophenol in the presence of Pd2(dba)3, t-BuOK and DPEphos in toluene at 100° C. to give thioether. Subsequent N-deprotection of this compound with HBr in refluxing H₂O obtains vortioxetine.

Large scale manufacturing of vortioxetine has been disclosed in WO 2010/094285. Piperazine, 2,4-dimethylthiophenol and 1,2-dihalogenbenzene are mixed e.g. in toluene together with a palladium catalyst to afford vortioxetine. Although this reaction provides high yield and can be handled in large scale, it requires the use of an expensive catalyst, i.e. palladium and a ligand. Moreover, the reaction conditions are stringent and employ elevated temperatures to obtain a satisfactory result, i.e. reflux temperatures of 80-120° C. and the use of strong base.

Each of these processes involves the use of a palladium catalyst and a phosphine ligand.

WO 2014/128207 discloses a one pot synthesis of vortioxetine hydrobromide which involves complexation of 1,2-dichlorobenzene with ferrocene in the presence of AlCl₃ and Al at 110° C., followed by treatment with NH₄PF₆ to yield eta(6)-1,2-dichlorobenzene-eta(5)-cyclopentadienyliron (II) hexafluorophosphate (II) which upon substitution with piperazine in the presence of K₂CO₃ in THF and optionally H₂O leads to phenyl piperazine derivative. Condensation of intermediate phenyl piperazine derivative with 2,4-dimethylthiophenol, generates thioether, which upon decomplexation by means of irradiation with light provides vortioxetine.

The preparation of vortioxetine is also described by Bang-Andersen et al. in J. Med. Chem. (2011), Vol. 54, 3206-3221. Here, in a first step, tert-butyl 4-(2-bromophenyl)piperazine-1-carboxylate intermediate is prepared from Boc-piperazine and 2-bromoiodobenzene in a palladium catalyzed coupling reaction. tert-Butyl 4-(2-bromophenyl)piperazine-1-carboxylate is then reacted with 2,4-dimethylthiophenol, again in the presence of palladium catalyst and a phosphine ligand, to provide Boc-protected vortioxetine. In the final step, vortioxetine is deprotected using hydrochloric acid to give vortioxetine hydrochloride.

WO-2014161976, CN-103788020, CN-103788019 disclose several related methods to prepare intermediate diaryl sulfides. Cross coupling of 2-nitrobenzenethiol with 1-bromo-2,4-dimethylbenzene using Pd₂(dba)₃, BINAP and t-BuOK in toluene at 100° C. or CuI and t-BuONa in acetonitrile irradiated by a mercury lamp gives thioether. This is alternatively obtained by cross coupling of 1-bromo-2-nitrobenzene with 2,4-dimethylthiophenol in the presence of Pd₂(dba)₃, BINAP and t-BuOK in toluene at 100° C. or CuI and t-BuONa in acetonitrile irradiated by a mercury lamp. Similarly, coupling of 1-halo-2-nitrobenzenes or with 2,4-dimethylthiophenol in the presence of K₂CO₃ in DMF produces 2,4-dimethyl-1-[(2-nitrophenyl)thio]benzene; followed by reduction of the nitro group in intermediate to the corresponding amine. Alternatively, intermediate diaryl sulfide can be obtained by Buchwald-Hartwig cross coupling of 1-iodo-2,4-dimethylbenzene with 2-aminobenzenethiol or 2-iodoaniline with 2,4-dimethylthiophenol using Pd₂(dba)₃, BINAP and t-BuOK in toluene at 100° C. Cyclization of 2-[(2,4-dimethylphenyl)thio]aniline with N,N-bis(2-chloroethyl)amine or the corresponding HCl salt or with N,N-bis(2-bromoethyl)amine in the presence of DIEA and KI in DMF at 75° C. or in DGME at 130° C. produces vortioxetine or its HCl salt. Finally, treatment of free base (or the corresponding hydrochloride previously basified with NaOH in H₂O/2-MeTHF) with HBr in i-PrOAc generates the desired vortioxetine hydrobromide.

Each of the above processes to prepare aryl sulfides has disadvantages. The process described in WO 2003/029232 is low yielding and unsuitable for the large scale production of serotonin reuptake inhibitors vortioxetine and tedatioxetine, whereas the processes described in WO 2007/144005 A1, WO-2014161976 and by Bang-Andersen et al. require the use of expensive starting materials, palladium catalyst and phosphine ligand. In addition, the toxicity of palladium is well known, Liu et al., Toxicity of Palladium, Toxicology Letters, 4 (1979) 469-473, and the European Medicines Agency's Guideline on the “Specification for Residues of Metal Catalysts” sets clear limits on the permitted daily exposure to palladium arising from palladium residue within drug substances, www.ema.europa.eu.

To overcome the drawbacks of the prior art, a process to prepare aryl halides is disclosed in WO-2014191548 wherein, thiol is condensed with diflurobenzene or 1-chloro-2-fluoro benzene in the presence of K₂CO₃ or Cs₂CO₃ in DMF at 100° C. to yield the corresponding thioethers. The process avoids use of palladium catalyst and ligands, however the condensation requires 3-4 days which is not suitable and economical on large scale industrial synthesis.

Thus it would be desirable to avoid or at least minimize the use of a palladium catalyst in the synthesis of serotonin reuptake inhibitors such as vortioxetine and the subsequent purification steps required to remove palladium residue from the final pharmaceutical product.

### OBJECTS OF THE INVENTION

An object of the present invention is to provide an improved process for preparing serotonin reuptake inhibitors of formula (I), including vortioxetine and tedatioxetine, and pharmaceutically acceptable salts and intermediates thereof.

Yet another object of the present invention is to provide a process which is simple, economical and suitable for industrial scale-up.

### SUMMARY OF THE INVENTION

The present invention provides a manufacturing process for serotonin reuptake inhibitors such as vortioxetine and tedatioxetine which uses inexpensive reagents, which can be run at mild conditions and which gives high yields relative to known processes.

Accordingly, the present invention relates to an improved process preparing serotonin reuptake inhibitors of formula (I) and pharmaceutically acceptable salts and intermediates thereof. The said process comprises:
coupling an aryl halide of formula VI:

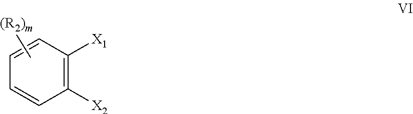

with a thiophenol of formula V:

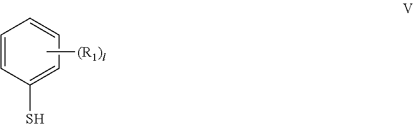

in the presence of copper catalyst and a base to form a compound of formula IV:

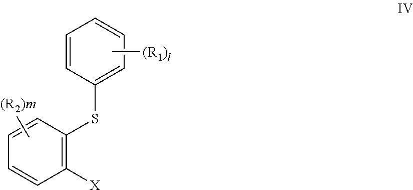

wherein R₁, R₂, l and m are as defined in relation to a compound of formula (I);
X₁ and X₂ which may be same or different, are independently selected from —H, halogen
and a protected piperazine group (Pg), provided that at least one of X₁ and X₂ is halogen;
X is selected from halogen and a protected piperazine group (Pg);
   and, thereafter, converting the compound of formula IV so formed into a compound of formula (I) or a pharmaceutically acceptable salt thereof.

Preferably, R₁ represents H or C₁₋₆ alkyl, more preferably H or methyl.

Preferably, R₂ represents H.

Preferably, l represents 1 or 2.

Preferably, m and n represent 0.

In a preferred aspect of the present invention, a compound of formula IV, wherein X represents halogen, is reacted with an optionally protected piperazine of formula III:

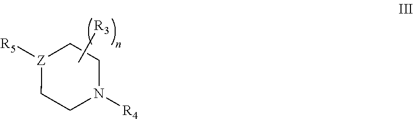

wherein Z, R₃ and n are as defined in relation to a compound of formula (I);
R₄ is selected from H and a protecting group (Pg); and
R₅ is H;
   in the presence of palladium catalyst to obtain a compound of formula II:

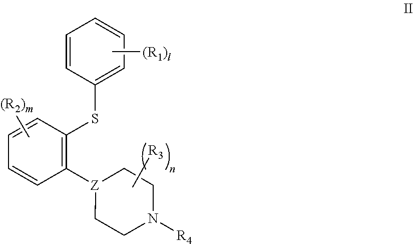

wherein R₁, R₂, R₃, Z, l, m and n are as defined in relation to a compound of formula (I)
and R₄, is selected from H and a protecting group (Pg).

If compound II is a protected piperazine, it may be subsequently deprotected by the addition of a suitable deprotecting agent, to obtain a compound of Formula (I):

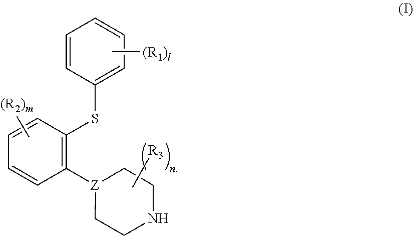

In another aspect of the present invention, the compound of formula (I) may be converted to a pharmaceutically acceptable salt form.

### DETAILED DESCRIPTION OF THE INVENTION

In one aspect of the present invention, there is provided an improved process for preparing serotonin reuptake inhibitors of Formula (I) and pharmaceutically acceptable salts and intermediates thereof as depicted in reaction Scheme 1.

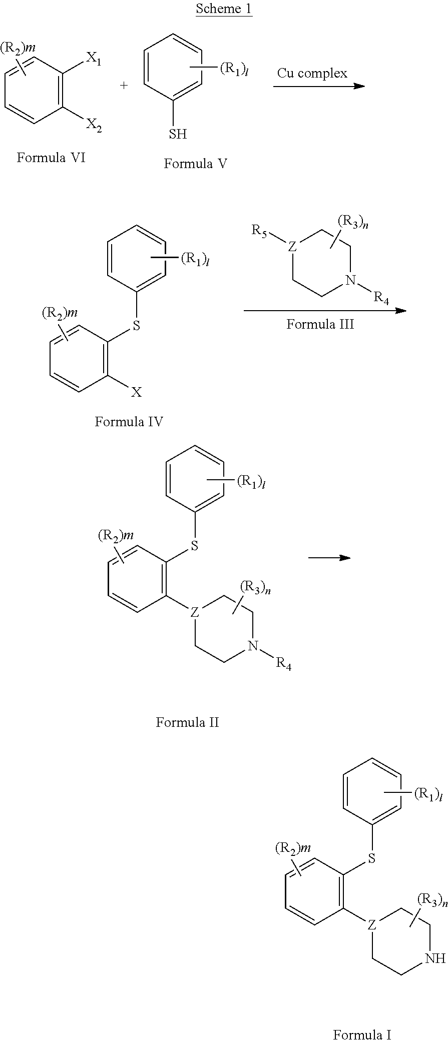

wherein R₁, R₂, R₃, R₄, R₅, Z, X₁, X₂, X, l, m and n are as hereinbefore defined.

Preferably, R₁ represents H or C₁₋₆ alkyl, more preferably H or methyl.

Preferably, R₂ represents H.

Preferably, l represents 1 or 2.

Preferably, m and n represent 0.

Preferably, X represents halogen.

Preferably, R₄ is selected from H and a protecting group (Pg).

Preferably, R₅ is H;

As used herein, the term “protecting group” (Pg) represents any amino protecting group, preferably a hydrolytically cleavable amino protecting group, selected from unsubstituted or substituted tert-carbyl, alkanoyl, arenecarbonyl, alkanesulfonyl, alkyloxycarbonyl, aryloxycarbonyl. More particularly, the “protecting group” represents a generally accepted protecting group, such as trityl (Tr), methanesulfonyl (Ms), p-toluenesulfonyl (Ts) or tert-butyloxycarbonyl (Boc). Preferably, the protecting group is tert-butyloxycarbonyl (Boc).

As used herein, the term “halogen” represents a halide ion, in particular, fluroide, chloride, bromide and iodide.

A feature of the present invention is the coupling of an aryl halide of formula VI with a thiophenol of formula V in the presence of a copper catalyst and a base to form a compound of formula IV.

Copper catalyzed C—S bond formation is efficient and operationally a simple reaction. Copper salts are very effective as catalysts providing the expected coupling products. The classical Cu-catalyzed reaction between thiols and aryl halides required stoichiometric amounts of copper salts, polar solvents and high temperature. In the context of the present invention, copper (I) salts are preferred catalysts over copper (II) salts in terms of conversion and yield, although either may be used in practice. Copper salts may be selected from the group comprising copper iodide, copper bromide, copper chloride or copper acetate. More preferably, the copper salt is copper iodide (CuI) due to its stability to air. This forms one aspect of the present invention. Preferably, the copper catalyst is present in the coupling reaction in an amount ranging from about 0.5 to about 10 mole %, such as from about 1 to about 10 mole %.

Many other Pd based catalytic systems, which are based on bidentate phosphines or diverse organophosphane derivatives, may be used in the coupling reaction and have been reported previously. However, these known systems have limitations since they require the preparation and use of trialkylphosphine (PR₃) ligands which are not eco-friendly. Catalytic systems based on other transition metals such as nickel, cobalt and iron also suffer from certain disadvantages, including metal toxicity, low turnover numbers and the like. Copper catalysis has an indisputable advantage over the other catalytic systems due to its low cost and the use of readily accessible and stable ligands. This forms another aspect of the present invention.

The reaction between an aryl halide of formula VI with a thiophenol of formula V may be enhanced by using various suitable copper ligands such as phosphazene P2-Et base, benzotriazole, trans-1,2-diaminocyclohexane, neocuproine and the like, primarily due to the high stability and low cost of copper. Preferably, neocuproine is used as a selective chelating agent to enhance the rate of the coupling reaction according to one aspect of the present invention. Preferably, the ligand is present in an amount ranging from about 0.1 to 10 about mole %.

A suitable base for use in the reaction between an aryl halide of formula VI with a thiophenol of formula V may be an inorganic or organic base. The inorganic base may be selected from the group consisting of alkali or alkaline earth metal carbonates, such as cesium carbonate, sodium carbonate, potassium carbonate, magnesium carbonate, calcium carbonate or barium carbonate; alkali or alkaline earth metal hydroxides such as sodium hydroxide, potassium hydroxide, lithium hydroxide, magnesium hydroxide, calcium hydroxide or barium hydroxide; alkoxides such as sodium t-butoxide, potassium t-butoxide, metal phosphates, such as monopotassium phosphate, dipotassium phosphate, tripotassium phosphate or any combination thereof. Organic bases may be aliphatic or aromatic and may be selected from, but not limited to triethyl amine, di-isopropyl amine, pyridine, picoline, diethyl amine, piperidine, N,N-diisopropylethylamine, 1,8-diazabicyclo [5.4.0]undec-7-ene (DBU) or any combination thereof. Preferably, the base is sodium t-butoxide.

Preferably, the coupling reaction between an aryl halide of formula VI with a thiophenol of formula V is performed using about 1-10 mole-% CuI, 0.1-10 mole-% neocuproine, with NaOtBu as the base to give aryl sulfides in excellent yields. The coupling reaction is preferably performed in the presence of an inert solvent under a nitrogen or argon atmosphere.

The solvent used for the reaction between an aryl halide of formula VI with a thiophenol of formula V may be selected from polar aprotic solvents such as dimethyl formamide, dimethyl sulfoxide, tetrahydrofuran, 1,4-dioxane, trioxane, N-methyl pyrrolidone, dimethyl acetamide; or ketones such as acetone, ethyl methyl ketone, methyl isobutyl ketone, methyl vinyl ketone; nitriles such as acetonitrile, propionitrile; ethers such as dimethoxyethane; polar protic solvents such as alcohols such as methanol, ethanol, isopropanol, t-butanol, t-amyl alcohol; optionally substituted hydrocarbon such as, mono ethylene glycol, toluene, xylene or any combination thereof.

Preferably, the reaction is performed in mono ethylene glycol. More preferably, the use of mono ethylene glycol in alcohol such as isopropanol serves as a co-solvent and a ligand in the reaction. The purpose of the co-solvent is to keep the copper catalyst (e.g. CuI) in solution.

Similar results are obtained with DME, DMF or dioxane in the absence of any additional ligand.

The temperature at which the reaction between an aryl halide of formula VI with a thiophenol of formula V proceeds is typically in the range of 30 to 120° C., preferably 50 to 120° C., more preferably 70 to 120° C., most preferably 80 to 120° C.

The reaction between an aryl halide of formula VI with a thiophenol of formula V is carried out at a time ranging from 1 hour to 24 hours, preferably 4 hours to 12 hours, most preferably 5 hours to 10 hours.

In one aspect of the present invention, an aryl halide of formula VI may be coupled with a thiophenol of formula V in the presence of 0.5 mol % CuI and 1 mol % benzotriazole in DMSO at 100° C. affording the sulfides in >90% yield.

In another aspect of the present invention, an aryl halide of formula VI may be coupled with a thiophenol of formula V in water at 120° C. in the presence of CuCl and trans-1,2-diaminocyclohexane.

In another aspect of the present invention, a compound of formula IV is reacted with a compound of formula III in the presence of a base, a solvent and a palladium catalyst consisting of a palladium source and a phosphine ligand; at a temperature between 60° C. and 130° C. to obtain a compound of formula II.

Useful palladium sources include palladium in different oxidations states, such as Pd(0) and Pd(II). Examples of palladium sources which may be used to catalyze the reaction between a compound of formula IV and a compound of formula III include, but are not limited to, Pd₂(dba)₃, Pd(dba)₂ and Pd(OAc)₂. The palladium source is typically applied in an amount of about 0.1-10 mole-%, such as about 1-10 mole-%, or about 1-5 mole-%.

As used herein, the term “mole-%” is calculated with respect to the limiting reactant.

Numerous phosphine ligands are known, both monedentate and bidentate, and may be employed in the process of the present invention. Examples of suitable phosphine ligands include, but are not limited to, racemic 2,2′-bis-diphenylphosphanyl-[1,1′]binaphtalenyl (rac-BINAP), 1,1′-bis(diphenyl phosphino)ferrocene (DPPF), bis(2-diphenylphosphinophenyl)ether (DPEphos), tri-t-butyl phosphine (Fu's salt), biphenyl-2-yl-di-t-butyl-phosphine, biphenyl-2-yl-dicyclohexyl-phosphine, (2′-dicyclohexylphosphanyl-biphenyl-2-yl)-dimethyl-amine, [2′-(di-t-butyl-phosphanyl)-biphenyl-2-yl]-dimethyl-amine, and dicyclohexyl-(2′,4′,6′-tri-propyl-biphenyl-2-yl)-phosphane.

Alternatively, carbene ligands, such as 1,3-bis-(2,6-di-isopropyl-phenyl)-3H-imidazol-1-ium chloride, may be used in the process of the present invention instead of phosphine ligands.

The reaction between a compound of formula IV with a compound of formula III is undertaken in the presence of a base. Examples of suitable bases include, but are not limited to, NaOt-Bu, KOt-Bu and Cs₂CO₃, 1,8-diazabicyclo [5.4.0]undec-7-ene (DBU) and 1,4-diazabicyclo [2.2.2] octane (DABCO), or any combination thereof. Typically, the base is added in an amount around 1-5 equivalents to the compound, preferably 1-3 equivalents, more preferably 2-3 equivalents.

If a protected piperazine of compound III or a protected aryl halide of formula VI or IXA is used in the reaction, then the protecting group has to be removed in a subsequent step, typically by the addition of aqueous acid to obtain a compound of formula (I).

The deprotection step involves removal of the protecting group using a suitable deprotecting agent. A suitable deprotecting agent is selected from an acid such as a strong mineral or organic acid, advantageously hydrofluoric acid, hydrochloric acid or trifluoroacetic acid; Lewis acid such as BF₃.ET₂O, zinc chloride or a suitable commercially available cationic resin such as DIAION™ SK110, TULSION™ T42H, and UBK558.

Compounds of formula (I) obtained by the process of the present invention, may be optionally, purified in a suitable solvent or mixture of solvents.

Compounds of formula (I) obtained by the process of the present invention may be further converted to pharmaceutically acceptable salts. Pharmaceutically acceptable salts are intended to indicate acid addition salts of acids that are non-toxic. Said salts include salts made from organic acids, such as maleic, fumaric, benzoic, ascorbic, succinic, oxalic, bis-methylenesalicylic, methanesulfonic, ethane disulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, theophylline acetic acids, as well as the 8-halotheophyllines, for example 8-bromotheophylline. Said salts may also be made from inorganic acids, such as hydrochloric, hydrobromic, sulfuric, sulfamic, phosphoric and nitric acids. Preferred salts are those made from hydrobromic acid, acetic acid and lactic acid.

In one preferred aspect, the compound obtained by the process of the invention is compound of formula (IA) or a pharmaceutically acceptable salt thereof:

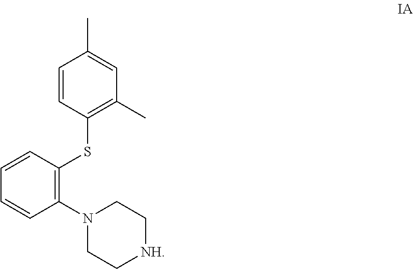 [SMILES: Cc1ccc(Sc2ccccc2N2CCNCC2)c(C)c1]

In another preferred aspect, the compound obtained by the process of the invention is a compound of formula (IB) or a pharmaceutically acceptable salt thereof:

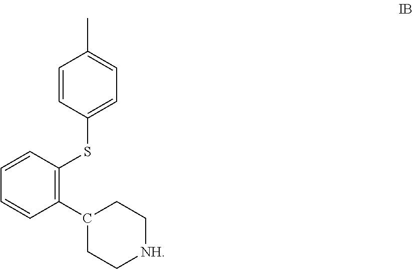 [SMILES: Cc1ccc(Sc2ccccc2C2CCNCC2)cc1]

Particularly preferred processes for preparing a compound of formula (IA) in accordance with the present invention are exemplified in Scheme 2.

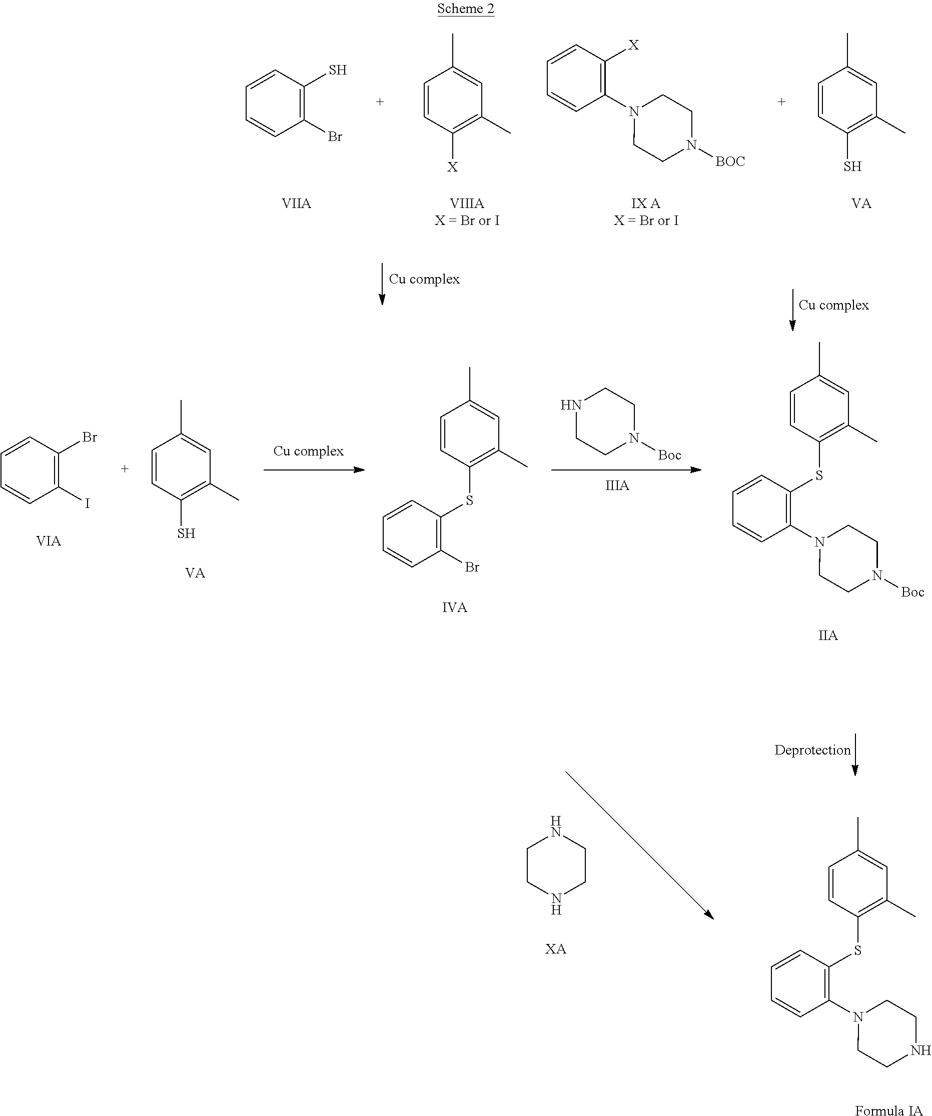

In one aspect of the invention, 1-bromo-2-iodo benzene (VIA) is coupled with 2,4-dimethl benzenethiol (VA) in presence of CuI and sodium t-butoxide, optionally using neocuproine in presence of an inert solvent such as toluene or monoethylene glycol to give intermediate compound 2-(2,4-dimethylphenyl sulfinyl) bromobenzene (IVA). The precursor (IVA) can be alternatively prepared by reaction of 2-bromo benzenethiol (VIIA) with 1-bromo-2,4-dimethylbenzene (VIIIA) in presence of CuI and sodium t-butoxide, optionally using neocuproine in presence of an inert solvent such as toluene or monoethylene glycol. Compound (IVA) is then reacted with N-Boc piperazine (IIIA) to give Boc protected vortioxetine (IIA). The precursor (IIA) can be alternatively prepared by reaction of a Boc protected aryl piperazine (IXA) with 2,4-dimethyl benzenethiol (VA) in presence of CuI and sodium t-butoxide, optionally using neocuproine in presence of an inert solvent such as toluene or monoethylene glycol, which is further deprotected with an acid to give vortioxetine (IA). Alternatively, 2-(2,4-dimethylphenyl sulfinyl) bromobenzene (IVA) is reacted with piperazine (XA) to produce vortioxetine. Vortioxetine obtained by the process of the present invention may be further reacted with aqueous HBr to prepare the corresponding hydrobromic acid addition salt.

In another aspect of the present invention, a modified process for preparing a compound of formula IA is as shown in Scheme 3.

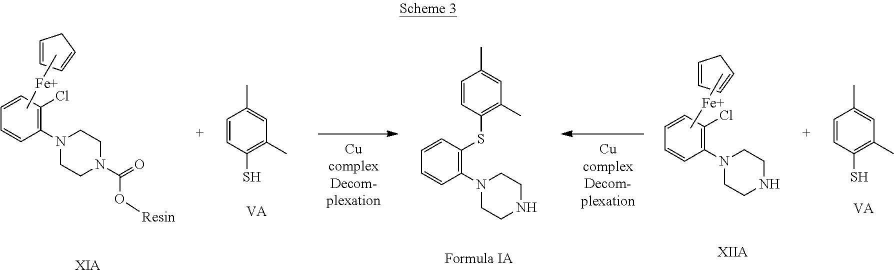

In accordance with still another aspect of the present invention, a modified process for preparing a compound of formula IA is as shown in Scheme 4.

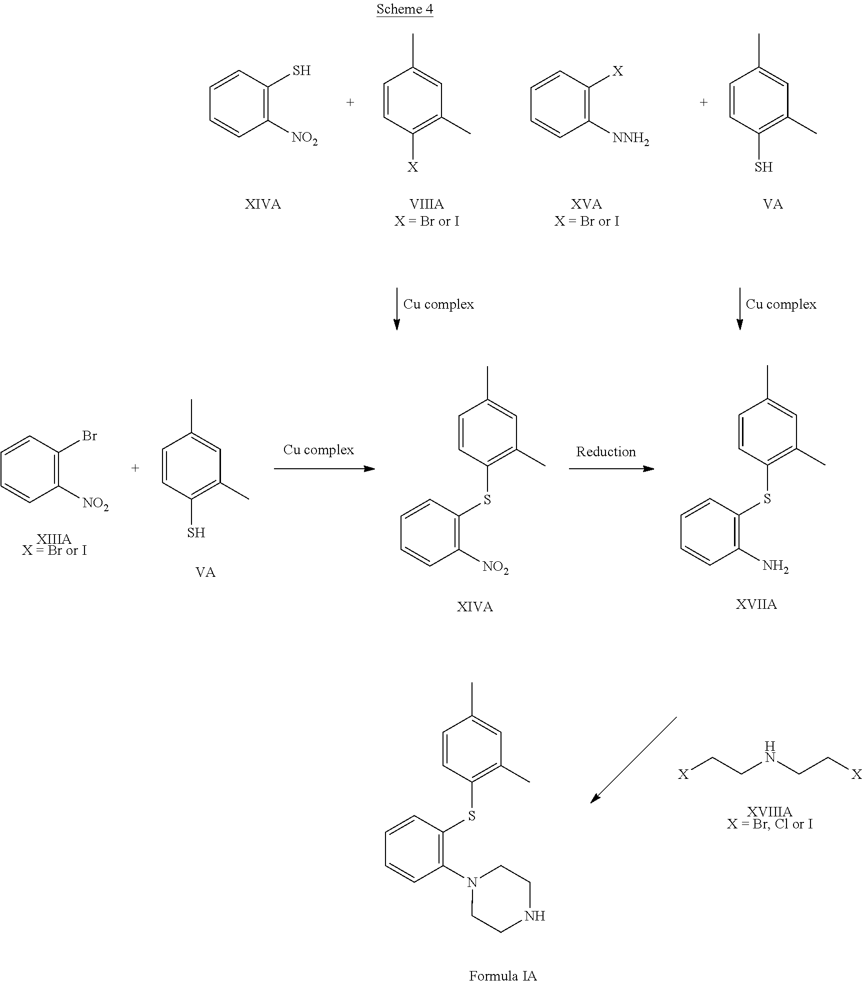

The following examples are non-limiting and serve to illustrate the invention.

### EXAMPLE 1

### 2-(2,4-dimethylphenyl sulfinyl) bromobenzene (compound IVA)

A mixture of lodo bromo benzene (10 g, 0.0353 moles), 2,4-dimethyl thiophenol (4.87 g, 0.0353 moles) were stirred in toluene (70 ml) under nitrogen. CuI (0.2 g, 0.00105 moles) and neocuproine (0.2 g, 0.000958 moles) and sodium t-butoxide (6.8 g, 0.0706 moles) were added. The reaction mixture was heated to 110° C. for 5-6 hours and then cooled to ambient temperature (r.t.). The reaction mixture was filtered and the clear organic layer was washed with water (2×50 ml). The organic layer was dried and the solvent was removed at reduced pressure to afford the title compound. Yield: 98-100%

### EXAMPLE 2

### 2-(2,4-dimethylphenyl sulfinyl) bromobenzene (compound IVA)

A mixture of lodo bromo benzene (10 g, 0.0353 moles), and monoethylene glycol (50 ml) were stirred in under nitrogen. CuI (0.2 g, 0.00105 moles) and neocuproine (0.02 g, 0.0000958 moles) and sodium t-butoxide (6.8 g, 0.0706 moles) were added. The reaction mixture was heated to 115° C. 2,4-dimethyl thiophenol (4.87 g, 0.0353 moles) was added and the reaction mixture was stirred for 5-6 hours at 110° C. The reaction mixture was cooled to ambient temperature (r.t.). Water (100 ml) and toluene (100 ml) were added and stirred for 15 minutes. The organic layer was separated, washed with water (50 ml) and the solvent was removed at reduced pressure to afford the title compound. Yield: 98-100%

### EXAMPLE 3

### 2-(2,4-dimethylphenyl sulfinyl) bromobenzene (compound IVA)

Mixture of sodium t-butoxide (6.8 g, 0.0706 moles) & monoethylene glycol (50.0 ml) were stirred under nitrogen for 15-30 mins. CuI (0.2 g, 0.00105 moles) & and iodo bromo benzene (10 g, 0.0353 moles) were added, under nitrogen at 25-30° C. The reaction mixture was heated to 115° C. 2,4-dimethyl thiophenol, (4.87 grams, 0.0353 moles) was added and the reaction mixture was stirred for 5-6 hours at 115° C. The reaction mixture was cooled to r.t. Water (100 ml) and toluene (100 ml) were added and stirred for 15 min. The organic layer was separated, washed with water (50 ml) and the solvent was removed at reduced pressure to afford the title compound. Yield: 98-100%

### EXAMPLE 4

### 2-(2,4-dimethylphenyl sulfinyl) bromobenzene (compound IVA)

A mixture of iodo bromo benzene (10 g, 0.0353 moles), and monoethylene glycol (50 ml) were stirred in under nitrogen. CuI (0.2 g, 0.00105 moles) and sodium t-Butoxide (6.8 g, 0.0706 moles) were added. The reaction mixture was heated to 115° C. 2,4-dimethyl thiophenol (4.87 grams, 0.0353 moles) was added and the reaction mixture was stirred for 5-6 hours at 115° C. The reaction mixture was cooled to ambient temperature (r.t.). Water (100 ml) and toluene (100 ml) were added and stirred for 15 min. The organic layer was separated, washed with water (50 ml) and the solvent was removed at reduced pressure to afford the title compound. Yield: 98-100%

### EXAMPLE 5

### Preparation of Vortioxetine Hydrobromide (Compound IA-HBr)

To a solution of compound IVA (100.0 g, 0.340 moles) in dry toluene (500 ml) was added at 25-30° C., N-Boc-Piperazine (75.5 g, 0.408 moles). Reaction mixture was purged by nitrogen for 30 mins. Pd₂(dba)₃ (5.0 g, 0.00546 moles) and racemic BINAP (5.0 g 0.00802 moles) were added at 25-30° C. Sodium tert-butoxide (65.3 g, 0.680 moles) was added and reaction mass heated to 110° C. for 6 hrs. Reaction mixture was cooled to 25-30° C., filtered and filtered cake was washed with toluene. Organic layer was washed with water and treated with charcoal.

Aqueous HBr was added to the organic layer at 25-30° C. Then reaction mixture was heated to 45-50° C. & stirred for 1-2 hrs. Reaction mixture was cooled 25-30° C. & stirred for 1 hr. Then product was isolated by filtration to afford the title compound. Yield: 100.0 g

1. A process for preparing a compound of formula (I), or a pharmaceutically acceptable salt thereof, 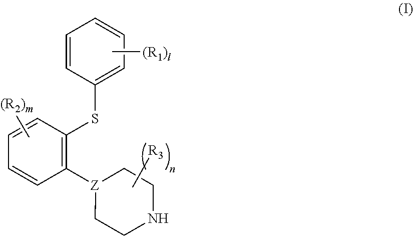
wherein Z is N, C or CH;
R₁ and R₂ are independently selected from a group represented by hydrogen, halogen, cyano, C₁₋₆-alkyl, C₁₋₆-alken, C₁₋₆-alkyn, C₁₋₆-alkenyl, C₁₋₆-alkynyl, C₁₋₆-alkyloxy, C₁₋₆-alkenyloxy, C₁₋₆-alkynyloxy, C₁₋₆-alkylylsulfanyl, C₁₋₆-alkenylsulfanyl, C₁₋₆-alkynylsulfanyl, hydroxy, hydroxy-C₁₋₆-alkyl, hydroxy-C₁₋₆-alken, hydroxy-C₁₋₆-alkyn, hydroxy-C₁₋₆-alkenyl, hydroxy-C₁₋₆-alkynyl, halo-C₁₋₆-alkyl, halo-C₁₋₆-alken, halo-C₁₋₆-alkyn, halo-C₁₋₆-alkenyl, halo-C₁₋₆-alkynyl, halo-C₁₋₆-alkyloxy, halo-C₁₋₆-alkenyloxy, halo-C₁₋₆-alkynyloxy, C₃₋₈-cycloalkyl, C₃₋₈-cycloalken, C₃₋₈-cycloalkenyl, C₃₋₈-cycloalk(en)yl-C₁₋₆-alk(en/yn) yl, C₁₋₆-alk (en/yn) ylsulfonyl, aryl, C₁₋₆-alkyloxycarbonyl C₁₋₆-alkenyloxycarbonyl C₁₋₆-alkynyloxycarbonyl, acyl, —NR₆CO—C₁₋₆-alkyl, —NR₆CO—C₁₋₆-alkenyl, —NR₆CO—C₁₋₆-alkynyl, CONR₆R₇ or NR₆R₇;
R₃ is independently selected from the group represented by C₁₋₆-alkyl, or two R₃ attached to the same carbon atom may form a 3-6-membered spiro-attached cycloalkyl; and wherein each R₆ and R₇ is independently selected from the group represented by hydrogen, C₁₋₆-alkyl, C₁₋₆-alkenyl, C₁₋₆-alkynyl, C₃₋₈-cycloalkyl, C₃₋₈-cycloalkenyl, C₃₋₈-cycloalk(en)yl-C₁₋₆-alk(en/yn)yl, or aryl; or R₆ and R₇ together with the nitrogen to which they are attached form a 3-7-membered ring which optionally contains one further hetero atom; provided that both R₆ and R₇ are not hydrogen;
l is 0, 1, 2, 3, 4 or 5;
m is 0, 1, 2, 3 or 4;
n is 0, 1, 2, 3, 4, 5, 6, 7 or 8;
comprising coupling an aryl halide of formula VI, 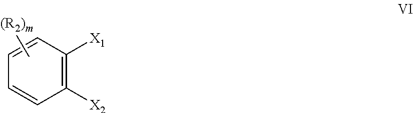
with a thiophenol of formula V, 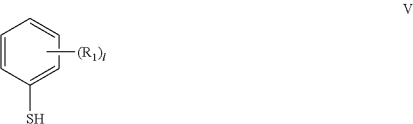
in the presence of a copper catalyst and a base to form a compound of formula IV, 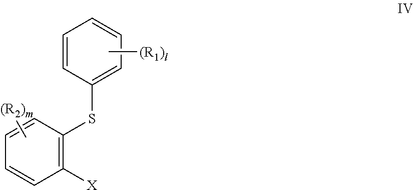
wherein, R₁, R₂, l and m are as defined in formula (I);
X₁ and X₂ which may be the same or different, are independently selected from H, halogen, and a protected piperazine group (Pg), provided that at least one of X₁ and X₂ is halogen; and
X is selected from halogen and a protected piperazine group (Pg);
and, thereafter, converting the compound of formula IV so formed into a compound of formula (I) or a pharmaceutically acceptable salt thereof.

2. A process according to claim 1, wherein the base is an inorganic base selected from the group consisting of alkali or alkaline earth metal carbonates, alkali or alkaline earth metal hydroxides, alkoxides and metal phosphates.

3. A process according to claim 2, wherein the inorganic base is selected from the group consisting of cesium carbonate, sodium carbonate, potassium carbonate, magnesium carbonate, calcium carbonate, barium carbonate, sodium hydroxide, potassium hydroxide, lithium hydroxide, magnesium hydroxide, calcium hydroxide, barium hydroxide, sodium t-butoxide, potassium t-butoxide, monopotassium phosphate, dipotassium phosphate and tripotassium phosphate, or any combination thereof.

4. A process according to claim 1, wherein the base is an organic base selected from the group consisting of triethyl amine, di-isopropyl amine, pyridine, picoline, diethyl amine, piperidine, N,N-diisopropylethylamine and 1,8-diazabicyclo [5.4.0]undec-7-ene (DBU), or any combination thereof.

5. A process according to claim 1, wherein the copper catalyst comprises a Cu(I) or Cu(II) salt.

6. A process according to claim 5, wherein the copper catalyst comprises copper iodide (CuI).

7. A process according to claim 1, wherein the copper catalyst is present in an amount ranging from about 0.5 mole % to about 10 mole %.

8. A process according to any claim 1, wherein the coupling reaction is performed in the presence of a solvent.

9. A process according to claim 8, wherein the solvent is a polar aprotic solvent, a polar protic solvent, an optionally substituted hydrocarbon, or any combination thereof.

10. A process according to claim 9, wherein the solvent is selected from dimethyl formamide, dimethyl sulfoxide, tetrahydrofuran, 1,4-dioxane, trioxane, N-Methyl pyrrolidone, dimethyl acetamide, acetone, ethyl methyl ketone, methyl isobutyl ketone, methyl vinyl ketone, acetonitrile, propionitrile, dimethyoxyethane methanol, ethanol, isopropanol, t-butanol, t-amyl alcohol, mono ethylene glycol, toluene, xylene, or any combination thereof.

11. A process according to claim 10, wherein the coupling reaction is performed at a temperature in the range of from about 30 to about 120° C.

12. A process according to claim 1, wherein the coupling reaction is performed in the presence of a ligand capable of acting as a selective chelating agent.

13. A process according to claim 12, wherein the ligand is present in an amount ranging from about 0.1 to about 10 mole %.

14. A process according to claim 12, wherein the ligand is phosphazene-P2-Et base, benzotriazole, trans-1,2-diaminocyclohexane, or neocuproine.

15. A process according to claim 14, wherein the ligand is neocuproine.

16. A process according to claim 1, wherein the coupling reaction is performed under a nitrogen or argon atmosphere.

17. A process according to claim 1 which further comprises the step of reacting a compound of formula IV, 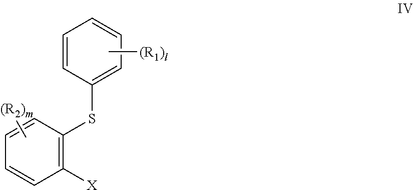
wherein R₁, R₂, l and m are as defined in formula (I) and X is a halogen with an optionally protected piperazine of formula III, 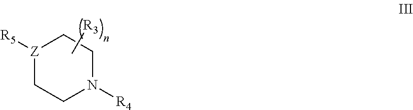
wherein, Z, R₃ and n are as defined in claim 1;
R₄ is H or a protecting group (Pg); and
R₅ is H;
in the presence of a palladium catalyst to obtain a compound of formula II, 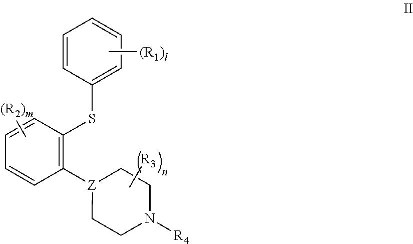
wherein, R₁, R₂, R₃, R₄, Z, l, m and n are as defined in claimed 1.

18. A process according to claim 1, which comprises removing any protecting group present to obtain a compound of formula (I), and optionally, thereafter, converting the compound of formula (I) so formed into a pharmaceutically acceptable salt thereof.

19. A process according to claim 18, wherein the protecting group is removed by treating a compound of formula II with a deprotecting agent which is selected from a mineral acid, an organic acid, a Lewis acid, or a cationic resin.

20. A process according to claim 1, wherein the protecting group is a hydrolytically cleavable amino protecting group.

21. A process according to claim 20, wherein the protecting group is selected from trityl, methanesulfonyl, p-toluenesulfonyl and tert-butyloxycarbonyl protecting groups.

22. A process according to claim 17, wherein the palladium catalyst comprises a Pd(0) or P(II) complex.

23. A process according to claim 22, wherein the palladium catalyst is selected from the group consisting of Pd₂(dba)₃, Pd(dba)₂ and Pd(OAc)₂.

24. A process according to claim 22, wherein the palladium catalyst is present in an amount ranging from about 0.1 to about 10 mole-%.

25. A process according to claim 17, wherein the reaction between the compound of formula IV and optionally protected piperazine of formula III is performed in the presence of a phosphine ligand.

26. A process according to claim 25, wherein the phosphine ligand is selected from the group consisting of racemic 2,2′-bis-diphenylphosphanyl-[1,1′]binaphtalenyl, 1,1′-bis(diphenyl phosphino)ferrocene, bis(2-diphenylphosphinophenyl)ether (DPEphos), tri-t-butyl phosphine, biphenyl-2-yl-di-t-butyl-phosphine, biphenyl-2-yl-dicyclohexyl-phosphine, (2′-dicyclohexylphosphanyl-biphenyl-2-yl)-dimethyl-amine, [2′-(di-t-butyl-phosphanyl)-biphenyl-2-yl]-dimethyl-amine and dicyclohexyl-(2′,4′,6′-tri-propyl-biphenyl-2-yl)-phosphane.

27. A process according to claim 17, wherein the reaction between the compound of formula IV and optionally protected piperazine of formula III is performed in the presence of a base.

28. A process according to claim 27, wherein the base is selected from the group consisting of NaOt-Bu, KOt-Bu, Cs₂CO₃, 1,8-diazabicyclo [5.4.0]undec-7-ene (DBU) and 1,4-diazabicyclo [2.2.2] octane (DABCO), or any combination thereof.

29. A process according to claim 27, wherein the base is present in an amount selected from 1-5 molar equivalents.

30. A process according to claim 17, wherein the reaction between the compound of formula IV and optionally protected piperazine of formula III is performed at a temperature in the range from about 60° C. to about 130° C.

31. A process according to claim 1, wherein the compound of formula (I) is 1-[2-(2,4-Dimethylphenylsulfanyl)phenyl]piperazine of formula IA,

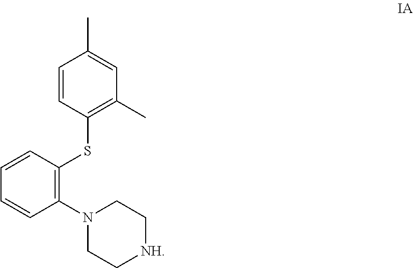 [SMILES: Cc1ccc(Sc2ccccc2N2CCNCC2)c(C)c1]

32. A process according to claim 1, wherein the compound of formula (I) is 4-{2-[(4-methylphenyl)sulfanyl]phenyl}piperidine of formula IB,

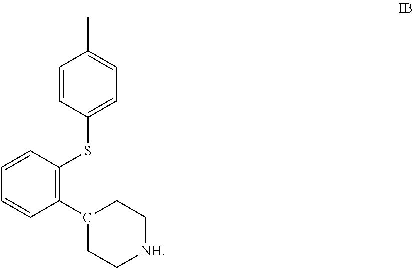 [SMILES: Cc1ccc(Sc2ccccc2C2CCNCC2)cc1]

33. A process according to claim 1, wherein the compound of formula VI is 1-bromo-2-iodo benzene (VIA),

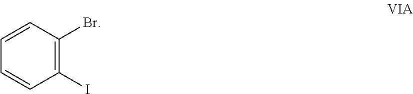

34. A process according to claim 1, wherein the compound of formula VI is 1-bromo-2,4-dimethylbenzene or 1-iodo-2,4-dimethylbenzene (VIIIA), 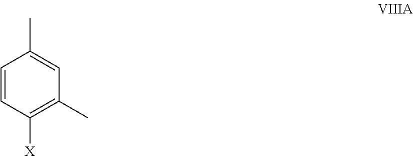
X=Br or I.

35. A process according to claim 1, wherein the compound of formula V is 2,4-dimethyl benzenethiol (VA),

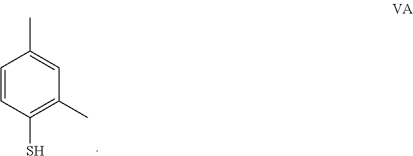

36. A process according to claim 1, wherein the compound of formula V is 2-bromo benzenethiol (VIIA),

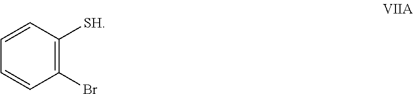

37. A process according to claim 1, wherein the compound IV is 2-(2,4-dimethylphenyl sulfinyl) bromobenzene (IVA),

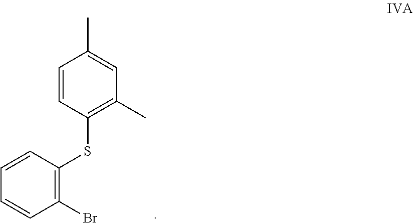 [SMILES: Cc1ccc(Sc2ccccc2Br)c(C)c1]

38. A process according to claim 17, wherein the compound of formula III is N-Boc piperazine (IIIA),

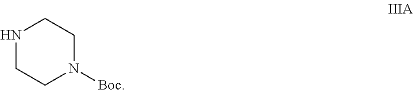 [SMILES: CC(C)(C)OC(=O)N1CCNCC1]

39. A process according to claim 17, wherein the compound of formula II is Boc protected vortioxetine (IIA),

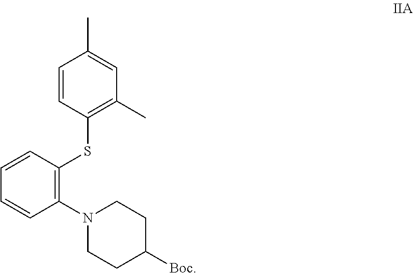 [SMILES: Cc1ccc(Sc2ccccc2N2CCC(C(=O)OC(C)(C)C)CC2)c(C)c1]

40. A process according to claim 1 wherein the process for preparing 1-[2-(2,4-Dimethylphenylsulfanyl)phenyl]piperazine of formula IA, 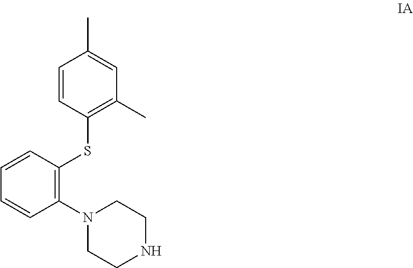 [SMILES: Cc1ccc(Sc2ccccc2N2CCNCC2)c(C)c1]
comprises the steps of,
coupling 1-bromo-2-iodo benzene (VIA), 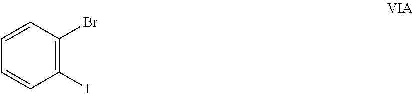
with 2,4-dimethyl benzenethiol (VA), 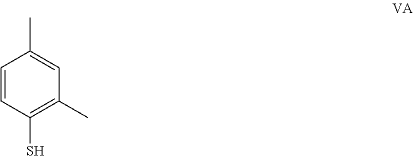
in the presence of a copper catalyst and a base to form the compound 2-(2,4-dimethylphenyl sulfinyl) bromobenzene (IVA), 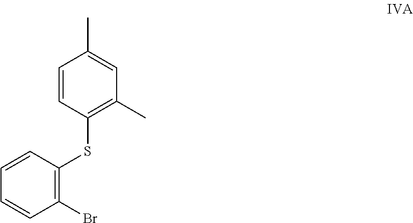 [SMILES: Cc1ccc(Sc2ccccc2Br)c(C)c1]
; and thereafter, either,
reacting the compound of formula (IVA) so formed with piperazine to obtain the compound of formula IA; or
reacting the compound of formula (IVA) so formed with the compound N-Boc piperazine (IIIA), 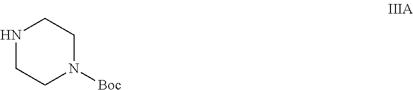 [SMILES: CC(C)(C)OC(=O)N1CCNCC1]
to obtain Boc-protected vortioxetine (IIA), 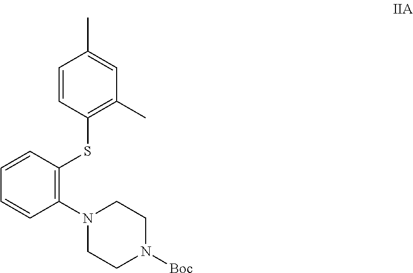 [SMILES: Cc1ccc(Sc2ccccc2N2CCN(C(=O)OC(C)(C)C)CC2)c(C)c1]
and thereafter deprotecting the compound of formula (IIA) so formed to obtain the compound of formula IA.

41. A process according to claim 1 wherein the process for preparing 1-[2-(2,4-Dimethylphenylsulfanyl)phenyl]piperazine of formula IA, 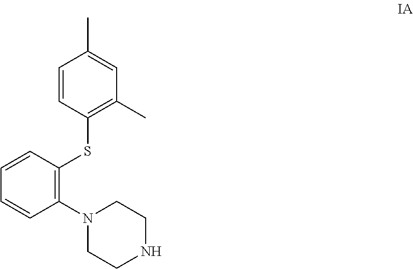 [SMILES: Cc1ccc(Sc2ccccc2N2CCNCC2)c(C)c1]
comprises the steps of,
coupling 2-bromo benzenethiol (VIIA), 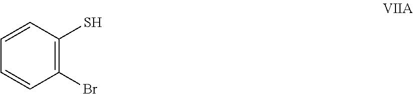
with 1-bromo-2,4-dimethylbenzene or 1-iodo-2,4-dimethylbenzene (VIIIA), 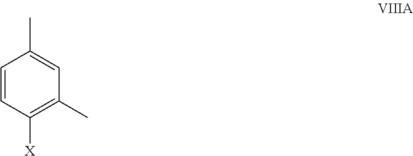
X=Br or I in the presence of a copper catalyst and a base to form the compound 2-(2,4-dimethylphenyl sulfinyl) bromobenzene (IVA), 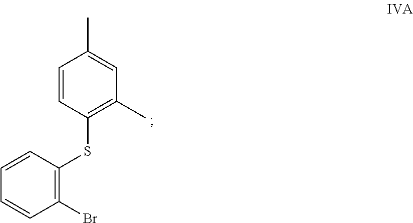 [SMILES: Cc1ccc(Sc2ccccc2Br)c(C)c1]
and thereafter, either,
reacting the compound of formula (IVA) so formed with piperazine to obtain the compound of formula IA;
or
reacting the compound of formula (IVA) so formed with the compound N-Boc piperazine (IIIA), 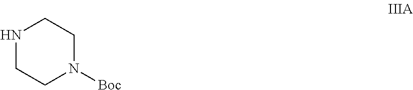 [SMILES: CC(C)(C)OC(=O)N1CCNCC1]
to obtain Boc-protected vortioxetine (IIA), 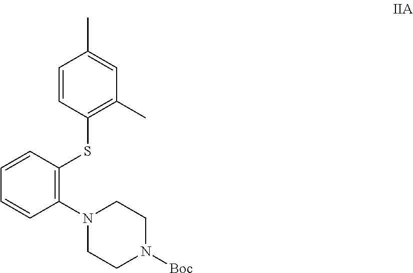 [SMILES: Cc1ccc(Sc2ccccc2N2CCN(C(=O)OC(C)(C)C)CC2)c(C)c1]
and thereafter deprotecting the compound of formula (IIA) so formed to obtain the compound of formula IA.

42. A process according to claim 1 wherein the process for preparing 1-[2-(2,4-Dimethylphenylsulfanyl)phenyl]piperazine of formula IA, 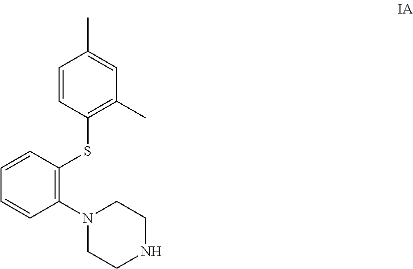 [SMILES: Cc1ccc(Sc2ccccc2N2CCNCC2)c(C)c1]
comprises the steps of, coupling a compound of formula IXA, 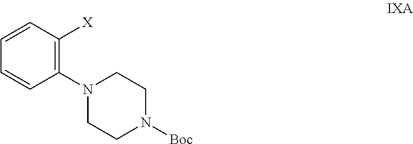
with a compound of formula VA, 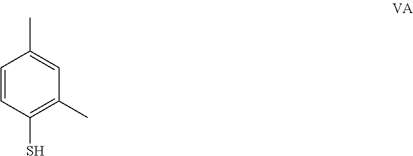
in the presence of a copper catalyst and a base to obtain Boc-protected vortioxetine (IIA), 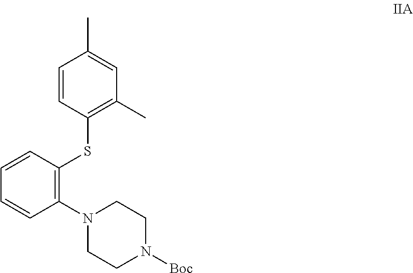 [SMILES: Cc1ccc(Sc2ccccc2N2CCN(C(=O)OC(C)(C)C)CC2)c(C)c1]
and thereafter deprotecting the compound of formula (IIA) so formed to obtain the compound of formula IA.

43. A process according to claim 39, further comprising the step of converting the compound of formula IA so formed into a pharmaceutically acceptable salt by treatment with a suitable acid.

44. A process according to claim 43, comprising treating the compound of formula IA with aqueous hydrobromic acid to obtain vortioxetine hydrobromide.

45. Vortioxetine or a pharmaceutically acceptable salt thereof prepared by a process according to claim 1.

46. Tedatioxetine or a pharmaceutically acceptable salt thereof prepared by a process according claim 1.

47. A pharmaceutical composition comprising vortioxetine or a pharmaceutically acceptable salt thereof prepared by a process according to claim 1 and one or more pharmaceutically acceptable excipients.

48. A pharmaceutical composition comprising tedatioxetine or a pharmaceutically acceptable salt thereof prepared by a process according to claim 1 and one or more pharmaceutically acceptable excipients.

49. Vortioxetine or a pharmaceutically acceptable salt thereof prepared substantially as described herein with reference to the Examples.

50. Tedatioxetine or a pharmaceutically acceptable salt thereof prepared substantially as described herein with reference to the Examples.
